(19) United States
(12) Patent Application Publication
(10) Pub. No.: US 2017/0108516 A1
(43) Pub. Date: Apr. 20, 2017

(54) DETECTION OF HEMOLYSIS USING A CHROMATOGRAPHIC DETECTION PAD

(71) Applicant: Siemens Healthcare Diagnostics Inc., Tarrytown, NY (US)

(72) Inventors: David J. Ledden, Medway, MA (US); Janine A. Cox, Stoughton, MA (US); Jeffrey R. Jasperse, Newton, MA (US)

(73) Assignee: Siemens Healthcare Diagnostics Inc., Tarrytown, NY (US)

(21) Appl. No.: 15/317,748

(22) PCT Filed: Jun. 8, 2015
(86) PCT No.: PCT/US15/34672

§ 371 Date: Dec. 9, 2016

Related U.S. Application Data
(60) Provisional application No. 62/011,633, filed on Jun. 13, 2014.

(51) Int. Cl. G01N 33/72; G01N 33/558

(52) U.S. Cl. CPC G01N 33/725; G01N 33/558; G01N 33/726

(57) ABSTRACT

In one aspect, the inventive concepts disclosed herein are directed to a chromatographic assay device for detecting the presence of free hemoglobin in a whole blood sample. The device comprising a chromatographic detection pad with a sample application site and a detection side. The chromatographic detection pad defines a path for capillary fluid flow. The chromatographic detection pad has a pore size. The sample application site on the chromatographic detection pad is for application of a portion of the whole blood sample. The detection site on the chromatographic detection pad is spaced apart from the application site and is downstream of the sample application site. The chromatographic detection pad is devoid of a compound located downstream of the application site that is reactive to the whole blood sample.

The subject application claims benefit under 35 USC §119(e) of U.S. provisional Application No. 62/011,633, filed Jun. 13, 2014. The entire contents of the above-referenced patent application are hereby expressly incorporated herein by reference.

### BACKGROUND

1. Field of the Disclosure

This disclosure relates to detecting hemolysis in a liquid sample using a chromatographic detection pad.

2. Brief Description of the Related Art

Hemolysis refers to the destruction or dissolution of red blood cells (RBCs) which results in the release of hemoglobin (“free hemoglobin”) into surrounding liquid. In the case of a whole blood sample, the free hemoglobin is released into the surrounding plasma. In the case of urine, the free hemoglobin is released into the surrounding water. The occurrence of hemolyzed RBCs may be the result of a patient's medical condition or by the mishandling the sample itself. When severe enough, hemolysis may result in inaccurate laboratory test results. For example, in blood gas and electrolyte testing it is known that hemolysis will cause an increase in the sample potassium level. In addition, it is known that cTnT levels are decreased in samples with hemolysis and cTnl levels have been shown to be increased in samples with hemolysis.

The detection of hemolysis in whole blood samples has traditionally been difficult. In a central laboratory setting, a whole blood sample is subjected to centrifugation—which generates plasma that is interrogated optically either in the near-infrared (NIR) or visible wavelength regions. While this technique is very effective, it is both complex and time consuming—thereby making this technique ineffective for Point of Care (POC) applications.

In the point of care arena some systems detect hemolysis electrochemically. However, electrochemical detection of hemoglobin and hematocrit is known to be inaccurate

### SUMMARY OF THE INVENTIVE CONCEPT(S)

In one aspect, the inventive concepts disclosed herein are directed to a chromatographic assay device for detecting the presence of free hemoglobin in a whole blood sample. The device comprising a chromatographic detection pad with a sample application site and a detection side. The chromatographic detection pad defines a path for capillary fluid flow. The chromatographic detection pad has a pore size. The sample application site on the chromatographic detection pad is for application of a portion of the whole blood sample. The detection site on the chromatographic detection pad is spaced apart from the application site and is downstream of the sample application site. The chromatographic detection pad is devoid of a compound located downstream of the application site that is reactive to the whole blood sample.

### BRIEF DESCRIPTIONS OF THE DRAWINGS

FIGS. 1 and 2 illustrate one embodiment of a chromatographic assay device.

FIG. 3 illustrates an embodiment of a medical diagnostics device.

### DETAILED DESCRIPTION OF THE INVENTIVE CONCEPT(S)

Before explaining at least one embodiment of the inventive concepts disclosed herein in detail, it is to be understood that the inventive concepts are not limited in their application to the details of construction and the arrangement of the components or steps or methodologies set forth in the following description or illustrated in the drawings. The inventive concepts disclosed herein are capable of other embodiments or of being practiced or carried out in various ways. Also, it is to be understood that the phraseology and terminology employed herein is for the purpose of description and should not be regarded as limiting the inventive concepts disclosed and claimed herein in any way.

In the following detailed description of embodiments of the inventive concepts, numerous specific details are set forth in order to provide a more thorough understanding of the inventive concepts. However, it will be apparent to one of ordinary skill in the art that the inventive concepts within the instant disclosure may be practiced without these specific details. In other instances, well-known features have not been described in detail to avoid unnecessarily complicating the instant disclosure.

As used herein, the terms “comprises,” “comprising,” “includes,” “including,” “has,” “having” or any other variation thereof, are intended to cover a non-exclusive inclusion. For example, a composition, a process, method, article, or apparatus that comprises a list of elements is not necessarily limited to only those elements but may include other elements not expressly listed or inherently present therein.

As used herein the terms “approximately,” “about,” “substantially” and variations thereof are intended to include not only the exact value qualified by the term, but to also include some slight deviations therefrom, such as deviations caused by measuring error, manufacturing tolerances, wear and tear on components or structures, settling or precipitation of cells or particles out of suspension or solution, chemical or biological degradation of solutions over time, stress exerted on structures, and combinations thereof, for example.

As used herein, the term “liquid sample” and variations thereof is intended to include, for example, but not limited to, biological fluids (such as urine and whole blood), chemical fluids, chemical substances, suspensions, solutions, slurries, mixtures, agglomerations, tinctures, slides, or other preparations of biological fluids, synthetic analogs to biological fluids, and combinations thereof.

Unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present). An inclusive or may be understood as being the equivalent to: at least one of condition A or B.

In addition, use of the “a” or “an” are employed to describe elements and components of the embodiments herein. This is done merely for convenience and to give a general sense of the inventive concepts. This description should be read to include one or at least one and the singular also includes the plural unless it is obvious that it is meant otherwise.

Finally, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

The inventive concepts disclosed herein are generally directed to a simple chromatographic assay device which uses a chromatographic detection pad (which may also be referred to as a lateral flow strip) for the detection of hemolysis in liquid samples to inform a medical professional when the sample is compromised and may yield inaccurate test results. The chromatographic assay device is able to rapidly detect hemolysis in a liquid sample 12 with a small sample size and at a low cost per test. Although this invention requires plasma separation (although not by centrifugation) it is fast and uses optical detection which is known to be more reliable.

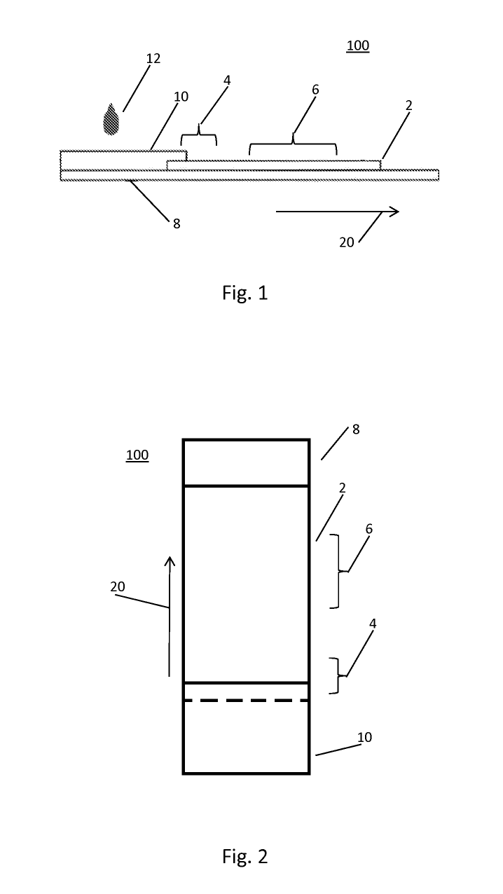

Referring now to FIGS. 1 and 2, a chromatographic assay device 100 for detecting the presence of free hemoglobin in a liquid sample 12, such as whole blood or urine, is shown. The chromatographic assay device 100 comprises a chromatographic detection pad 2 through which the liquid sample 12 flows through by capillary action (which may also be referred to as capillary flow). The chromatographic detection pad 2 may be made of any suitable material through which the liquid sample 12 may flow by capillary action. As an example, the chromatographic detection pad 2 may be a nitrocellulose membrane. The chromatographic detection pad 2 may have pores through which the liquid sample 12 moves by capillary action. The majority of the pores of the chromatographic detection pad 2 may all be substantially the same size or fall within a range of values. The chromatographic detection pad 2 may be attached to a backing material 8 via double stick adhesive.

The chromatographic detection pad 2 has a sample application site 4 and a detection site 6. Sample application site 4 is the area at which the liquid sample 12 comes into contact with the chromatographic detection pad 2. In FIGS. 1 and 2, sample application site 4 is adjacent to the end of the chromatographic detection pad 2 but it should be appreciated that this is merely one of several possible locations. As will be explained further below, the sample application site 4 may be treated with a red blood cell (RBC) binding or agglutination material.

Detection site 6 of the chromatographic detection pad 2 is spaced apart from the sample application site 4 such that the liquid sample 12 flows through the chromatographic detection pad 2 via capillary action from the sample application site 4 towards the detection site 6 in the direction of arrow 20. Thus the detection site 6 should be understood as being downstream of the sample application site 4.

As will be explained further below, the chromatographic detection pad 2 may be devoid of a compound located downstream of the sample application site 4 that is reactive to the liquid sample. Alternatively, the chromatographic detection pad 2 may contain one or more reagents that react with free hemoglobin present in the liquid sample 12 flowing through the chromatographic detection pad 2. An exemplary reagent present in the detection pad 2 may accentuate the color change attributable to free hemoglobin in the detection zone. Exemplary reagents utilize the peroxidase-like activity of hemoglobin, which catalyzes the reaction of diisopropylbenzene dihydroperoxide and 3,3′, 5,5′-tetramethylbenzidine. The resulting color ranges from orange through green and possibly up to blue. Exemplary reagents located in the detection pad 2 may be arranged into a strip arranged perpendicular to the direction of flow (which is denote by arrow 20).

The chromatographic assay device 100 may also contain an absorbent sample application pad 10 that is fluidic contact with the sample application site 4 of the chromatographic detection pad 2. The sample application pad 10 may receive and absorb the liquid sample 12. The liquid sample 12 may then be absorbed into the chromatographic detection pad 2 from the sample application pad 10 at sample application site 4. In various embodiments of the invention, the sample application pad 10 may or may not contain one or more red blood cell (RBC) binding or agglutination material. RBC binding or agglutination material may include, individually or in combination: (1) a human Red Blood Cell (hRBC) binding or agglutination protein; (2) a lectin binding or agglutination protein; or (3) an anti-human Red Blood Cell (anti-hRBC) binding or agglutination protein.

The pore size(s) of the chromatographic detection pad 2 and the presence of one or more RBC binding or agglutination materials are designed to allow primarily free hemoglobin to flow freely through the chromatographic detection pad 2 but not RBCs. Other components of the liquid sample 12, such as plasma in the case of whole blood, are also able to flow through the chromatographic detection pad 2. Individual RBCs have a diameter of approximately 7 microns. However, when the sample application site 4 and/or the sample application pad 10 contains one or more RBC binding or agglutination materials, the RBC binding or agglutination material(s) agglutinates RBCs in the liquid sample 12 together to produce agglutinated RBCs that are larger than individual RBCs. The size of agglutinated RBCs depends on the number of RBCs that have been joined together (e.g., two agglutinated RBCs have a size of approximately 14 microns, three agglutinated RBCs have a size of approximately 21 microns, and so on).

Thus, when a chromatographic detection pad 2 has a pore size of less than 7 microns, individual RBCs are not able to flow through the chromatographic detection pad 2 and RBC binding or agglutination material(s) are not required in order to ensure that primarily free hemoglobin and plasma flows through the chromatographic detection pad 2. In various embodiments, the sample application pad 10 and the sample application site 4 are devoid of RBC capture material(s) and the pore size(s) of the chromatographic detection pad 2 can be, for example, less than 2 microns, approximately 1 micron; approximately 0.45 microns; or approximately 0.22 microns.

However, when a chromatographic detection pad 2 has a pore size(s) of more than 7 microns (i.e., larger than an individual RBC), RBC binding or agglutination material(s) are utilized in order to prevent individual RBCs from flowing through the chromatographic detection pad 2. An exemplary pore size of more than 7 microns but less than 14 microns thereby prevents two or more agglutinated RBCs from flowing through chromatographic detection pad 2 while still allowing primarily free hemoglobin and plasma to flow through the chromatographic detection pad 2. In an embodiment, the pore size(s) of the chromatographic detection pad 2 is between 8 and 13 microns. While in order embodiment, the pore size the pore size(s) of the chromatographic detection pad 2 may be between 8 and 40 microns. In yet another embodiment, pore sizes of a chromatographic detection pad 2 may be between 2 microns and 40 microns—in which case the chromatographic detection pad 2 can contain RBC binding or agglutination material(s).

The pore size(s) of the chromatographic detection pad 2 determines the flow rate of the liquid sample 12 through the chromatographic detection pad 2. Larger pore sizes (e.g., above 8 microns) results in a higher flow and ultimately a faster test result. On the other hand, a chromatographic assay device 100 with smaller pore sizes (e.g., less than 2 microns) has in a slower flow rate but does not need RBC binding or agglutination material(s). The flow rate of chromatographic detection pad 2 may be used to determine how porous a chromatographic detection pad 2 is. Flow rate for a chromatographic detection pad 2 can be measured in sec/4 cm. The relationship between flow rate and pore size can vary by manufacturer.

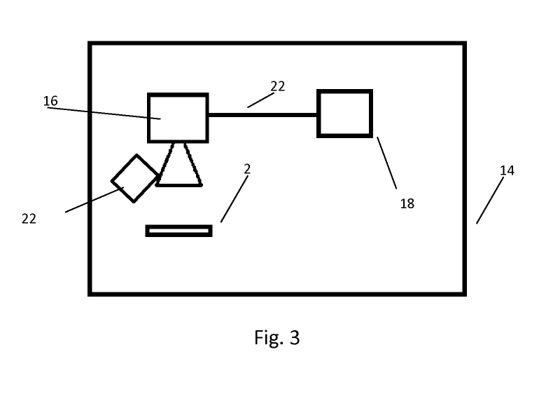

Referring now to FIG. 3, a medical diagnostics device 14 is depicted. Diagnostics device 14 comprises an optical sensor 16, a processor 18, and a light source 22 directed at the detection site 6. The optical sensor 16 takes one or more images of the detection site 6 and transmits the image(s) to the processor 18 in detection signal(s) 22. The processor 18 then analyzes the characteristics of the light reflected by the detection site 6 of the chromatographic detection pad 2 based on the received image(s). The characteristics, such as the observable colors (e.g., red, orange, green, and blue), of the light reflected by the detection site 6 are attributable to the presence of free hemoglobin in the liquid sample 12. Thus, the characteristics of the reflect light can be used by the processor to quantify the amount of free hemoglobin present in the sample liquid 12. For example, when the detection zone 6 is devoid of a compound located downstream of the sample application site 4, the amount/intensity of red light reflected by the detection site 6 can be used to quantify the amount of free hemoglobin present in the liquid sample 12. When the chromatographic detection pad 2 contains a reagent(s) that reacts with free hemoglobin and is located downstream of the sample application site 4, the amount/intensity of one or more of red, orange, green, or blue light reflected by the detection site 6 can be used to quantify the amount of free hemoglobin present in the liquid sample 12. Thus the processor 18 is able to determine the amount of free hemoglobin in the liquid sample 12 by, for example, comparing the measured amounts of the observable colors of the light reflected by the detection site 6 against known reference values. It should also be understood that the processor 18 need not be located within the device 14 and can be located at an external location.

In an embodiment, the light source 20 may be a broadband light source and the optical sensor 16 may employ a two dimensional array of pixels capturing a two dimensional image of the detection site 6. The processor 16 may be configured to select specific regions of interest within the image of the chromatographic assay substrate, analyze spectral content and surface topography of the regions of interest on the substrate, determine porosity and depth variation of the regions of interest, algorithmically improve selectivity, dynamic range, and signal to noise of the primary signals of interest, that are otherwise degraded by variations in the detection region, residual sample turbidity and chemical interferents.

A method of testing a liquid sample for hemolysis may include measuring the characteristics of the light reflected by the detection site 6 of the chromatographic assay 100, as described above, after a portion of the liquid sample 12 has been applied to the sample application site 4 and free hemoglobin has flowed into the detection site 6. The measured amount(s) of, for example, red, orange, green, and/or blue light can then be used in determining the level of free hemoglobin by, for example, comparing the measured amount(s) against one or more reference values. In exemplary embodiments, the method may be performed by device 14 or by a medical provider. A medical provider may, for example, compare the completed the chromatographic assay 100 against a reference device, containing reference colors which correspond to different levels of hemolysis, in order to visually determine the hemolysis of the liquid sample 12.

This method may be used to detect the levels of hemoglobin that exceed a predetermined interference value (for example a manufacturers' interference level). If the sample is above the interference value, the sample would be flagged to inform the end user (i.e., the relevant healthcare provider) that the sample is hemolyzed and therefore compromised. Where device 14 is able to perform additional tests on the liquid sample 12 after determining that the liquid sample 12 is hemolyzed, the device 14 may either prevent a subsequent test from being performed using the liquid sample 12 or allow a subsequent test to be performed using the liquid sample 12 but notify the end user to take into account that the liquid sample 12 is hemolyzed when interpreting the results of the subsequent test(s).

In one embodiment, a whole blood sample is applied to the sample application pad 10 containing RBC binding or agglutination material(s). Only if the whole blood sample is hemolyzed will free hemoglobin migrate (e.g., flow) from the sample application site 4 to the detection site 6 where the red color can be detected visually and/or instrumentally.

In another embodiment, a whole blood sample is applied to the chromatographic detection pad 2 (and/or the sample application pad 10) which has no additives and has a pore size of less than 2 microns.

Processor 18 may have any suitable architecture, such as a general processor, central processing unit, digital signal processor, application specific integrated circuit, field programmable gate array, digital circuit, analog circuit, combinations thereof, or any other now known or later developed device for processing data. Likewise, processing strategies may include multiprocessing, multitasking, parallel processing, and the like. A program may be uploaded to, and executed by, the processor. The processor implements the program alone or includes multiple processors in a network or system for parallel or sequential processing.

The processor outputs the state and/or associated information on the display, into a memory, over a network, to a printer, or in another media. The display is text, graphical, or other display.

The display is a CRT, LCD, plasma, projector, monitor, printer, or other output device for showing data. The display is operable to output to a user a state associated with a patient. The state provides an indication of whether a medical concept is indicated in the medical transcript. The state may be whether a disease, condition, symptom, or test result is indicated. In one embodiment, the state is limited to true and false, or true, false and unknown. In other embodiments, the state may be a level of a range of levels or other non-Boolean state.

The processor operates pursuant to instructions. The instructions may be embodied in a program. The program may be a non-transitory computer-readable medium that stores instructions that, when executed by the at least on processor 18 cause the processor 18 to quantify the amount of free hemoglobin present in the liquid sample 12 based on a image(s) of the detection site 6 according to any one of the techniques described herein. The program may be located non-transitory a computer readable memory such as an external storage, ROM, and/or RAM. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system. Because some of the constituent system components and method acts depicted in the accompanying figures may be implemented in software, the actual connections between the system components (or the process steps) may differ depending upon the manner of programming.

1. A chromatographic assay device for detecting the presence of free hemoglobin in a whole blood sample, the device comprising:
a chromatographic detection pad which defines a path for capillary fluid flow, the chromatographic detection pad having a pore size;
a sample application site on the chromatographic detection pad for application of a portion of the whole blood sample, the sample application site being adjacent to a first end of the chromatographic detection pad;
a detection site on the chromatographic detection pad, the detection spaced apart from the application site, the detection site being downstream of the sample application site; and
the chromatographic detection pad being devoid of a compound located downstream of the application site that is reactive to the whole blood sample.

2. The chromatographic assay device of claim 1, further comprising a sample application pad in fluidic contact with the sample application site of the chromatographic detection pad.

3. The chromatographic assay claim 2, wherein the sample application pad contains at least one type of red blood cell (RBC) binding or agglutination material, when the portion of the whole blood sample is placed on the sample application pad the RBC material agglutinates with any RBCs in the sample to produce agglutinated RBCs, the agglutinated RBCs having a size greater than the pore size of the chromatographic detection pad, the agglutinated RBCs thereby being prevented from flowing through the chromatographic detection pad.

4. The chromatographic assay device of claim 3, wherein the RBC binding or agglutination material comprises a human Red Blood Cell (hRBC) binding or agglutination protein.

5. The chromatographic assay device of claim 2, wherein the RBC binding or agglutination material comprises at least one of a lectin or an anti-human Red Blood Cell (anti-hRBC) binding or agglutination protein.

6. The chromatographic assay of claim 1, wherein the pore size of the chromatographic detection pad is between 8 and 13 microns, the pore size preventing agglutinated RBCs from flowing through the chromatographic detection pad.

7. The chromatographic assay of claim 1, wherein the pore size of the chromatographic detection pad prevents individual RBCs from flowing through the chromatographic detection pad.

8. The chromatographic assay of claim 1, wherein the pore size of the chromatographic detection pad is less than 2 microns.

9. The chromatographic assay of claim 1, wherein the sample application pad is devoid of RBC capture material.

10. A medical diagnostics device, the device comprising:
an optical sensor, the optical sensor detecting the amount of red light reflected by the detection site of the chromatographic assay of claim 1 and outputting a detection signal, the amount of red light reflected by the detection site being attributable to the presence of free hemoglobin in the whole blood sample, the free hemoglobin and plasma being able to flow through the chromatographic detection pad;
a processor, the processor receiving the detection signal and determining the amount of free hemoglobin in the whole blood sample.

11. A method of testing a liquid sample for hemolysis comprising:
measuring the amount of red light reflect by the detection site the amount of red light reflected by the detection site of the chromatographic assay of claim 1 after the portion of the sample of whole blood has been applied to the sample application site, the amount of red light reflected by the detection site being attributable to the presence of free hemoglobin in the whole blood sample, the free hemoglobin and plasma being able to flow through the chromatographic detection pad; and
determining the level of free hemoglobin based on the measured amount of reflected red light.

12. The method of claim 11, further comprising:
when the amount of reflected red light exceeds a predefined reference value, notifying a healthcare provider that the sample is hemolyzed.

13. The method of claim 12, further comprising:
preventing a subsequent test from being performed using the sample of whole blood.

14. The method of claim 11, further comprising:
when the amount of reflected red light does not exceed a predefined reference value, allowing a subsequent test to be performed using the sample of whole blood; and
reporting the results of the subsequent test to the healthcare provider.

15. The chromatographic assay of claim 1, wherein the chromatographic detection pad contains a reagent located downstream of the application site that is reactive to the free hemoglobin in the liquid sample.

16. The chromatographic assay of claim 15, wherein the reagent utilizes the peroxidase-like activity of hemoglobin, the peroxidase-like activity of hemoglobin catalyzing the reaction of diisopropylbenzene dihydroperoxide and 3,3′, 5,5′-tetramethylbenzidine.
